(19) United States
(12) Patent Application Publication
(10) Pub. No.: US 2026/0087794 A1
(43) Pub. Date: Mar. 26, 2026

(54) MEDICAL IMAGE PROCESSING APPARATUS, MEDICAL IMAGE PROCESSING SYSTEM, SERVER, MEDICAL IMAGE PROCESSING METHOD, AND COMPUTER-READABLE RECORDING MEDIUM

(71) Applicant: OLYMPUS MEDICAL SYSTEMS CORP., Tokyo (JP)

(72) Inventor: Masahide YAMAKI, Tokyo (JP)

(73) Assignee: OLYMPUS MEDICAL SYSTEMS CORP., Tokyo (JP)

(21) Appl. No.: 19/404,455

(22) Filed: Dec. 1, 2025

Related U.S. Application Data
(63) Continuation of application No. PCT/JP2023/041673, filed on Nov. 20, 2023.
(60) Provisional application No. 63/470,217, filed on Jun. 1, 2023.

(51) Int. Cl. G06V 10/94; A61B 1/00; G06T 7/00; G06V 40/20; G16H 30/40

(52) U.S. Cl. CPC G06V 10/95; A61B 1/00009; G06T 7/0012; G06V 40/20; G16H 30/40; G06T 2207/10068; G06V 2201/034

(57) ABSTRACT

A medical image processing apparatus includes: a processor including hardware, the processor being configured to: acquire, from an external server, at least one processing module corresponding to connected device information specific to a connected medical observation device for identifying the medical observation device and procedure information regarding procedure; and execute image processing on imaging data obtained by imaging using the medical observation device according to an image processing algorithm corresponding to the at least one processing module.

### CROSS REFERENCES TO RELATED APPLICATIONS

This application is a continuation of International Application No. PCT/JP2023/041673, filed on Nov. 20, 2023 which claims the benefit of priority of U.S. Provisional Application No. 63/470,217 filed on Jun. 1, 2023, the entire contents of which are incorporated herein by reference.

### BACKGROUND

### 1. Technical Field

The present disclosure relates to a medical image processing apparatus, a medical image processing system, a server, a medical image processing method, and a computer-readable recording medium.

### 2. Related Art

In the related art, a technology for causing a server to execute image processing on imaging data obtained by imaging using a medical observation device such as an endoscope is known (see, for example, JP 2022-42011 A).

In the technology described in JP 2022-42011 A, the medical observation device is communicably connected to the server and transmits the imaging data obtained by imaging to the server. Then, the server executes image processing on the received imaging data, and displays a display image corresponding to the imaging data after the image processing on a monitor.

### SUMMARY

In some embodiments, a medical image processing apparatus includes: a processor including hardware, the processor being configured to: acquire, from an external server, at least one processing module corresponding to connected device information specific to a connected medical observation device for identifying the medical observation device and procedure information regarding procedure; and execute image processing on imaging data obtained by imaging using the medical observation device according to an image processing algorithm corresponding to the at least one processing module.

In some embodiments, a medical image processing system includes: a server; and the medical image processing apparatus. The medical image processing apparatus is communicably connected to the server.

In some embodiments, a server includes: a communicator communicably connected to an external medical image processing apparatus; a controller configured to control an operation of the communicator; and a storage configured to store connected device information specific to a medical observation device for identifying the medical observation device and procedure information regarding procedure in association with at least one processing module. The controller is configured to read, from the storage, the at least one processing module associated with the connected device information and the procedure information acquired from the medical image processing apparatus via the communicator, and transmit the at least one processing module to the medical image processing apparatus via the communicator.

In some embodiments, provided is a medical image processing method executed by a medical image processing apparatus. The medical image processing method includes: acquiring, from an external server, at least one processing module corresponding to connected device information specific to a medical observation device for identifying the medical observation device connected to the medical image processing apparatus and procedure information regarding procedure; and executing image processing on imaging data obtained by imaging using the medical observation device according to an image processing algorithm corresponding to the at least one processing module.

In some embodiments, provided is a non-transitory computer-readable recording medium with an executable program stored thereon. The program causes a processor of a medical image processing apparatus to execute: acquiring, from an external server, at least one processing module corresponding to connected device information specific to a medical observation device for identifying the medical observation device connected to the medical image processing apparatus and procedure information regarding procedure; and executing image processing on imaging data obtained by imaging using the medical observation device according to an image processing algorithm corresponding to the at least one processing module.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a configuration of an endoscope system according to an embodiment;

FIG. 2 is a block diagram illustrating a configuration of a control device;

FIG. 3 is a block diagram illustrating a configuration of a server;

FIG. 4 is a diagram illustrating an example of a medical image processing method;

FIG. 5 is a diagram illustrating the example of the medical image processing method;

FIG. 6 is a diagram illustrating a first modification of the embodiment;

FIG. 7 is a diagram illustrating the first modification of the embodiment;

FIG. 8 is a diagram illustrating a second modification of the embodiment;

FIG. 9 is a diagram illustrating the second modification of the embodiment;

FIG. 10 is a diagram illustrating a third modification of the embodiment;

FIG. 11 is a diagram illustrating the third modification of the embodiment;

FIG. 12 is a diagram illustrating a fourth modification of the embodiment;

FIG. 13 is a diagram illustrating the fourth modification of the embodiment; and

FIG. 14 is a diagram illustrating a fifth modification of the embodiment.

### DETAILED DESCRIPTION

Hereinafter, a mode for carrying out the disclosure (embodiments) will be described with reference to the drawings. Note that the disclosure is not limited by the embodiments described below. Furthermore, in the drawings, the same parts are denoted by the same reference numerals.

### Schematic Configuration of Endoscope System

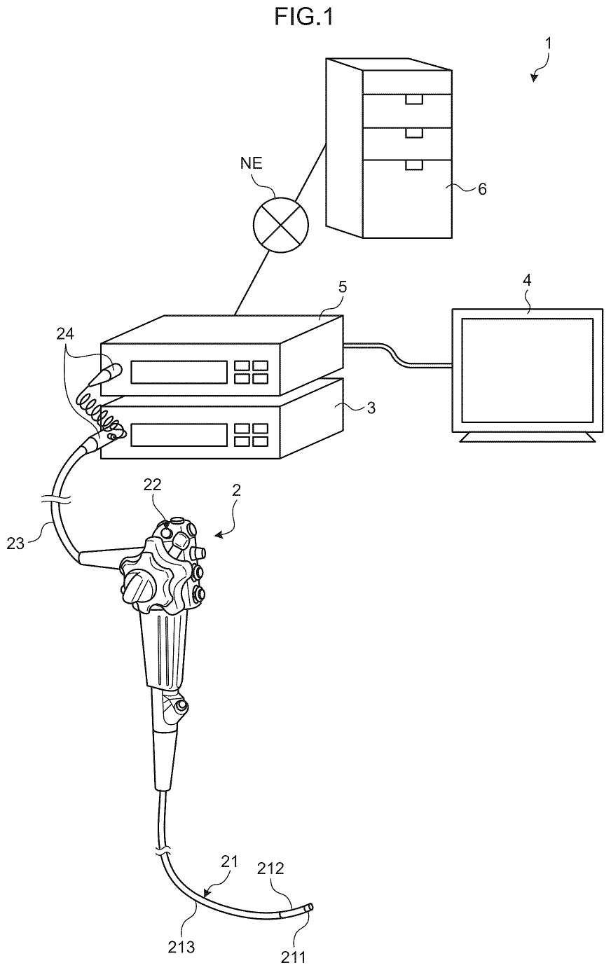

FIG. 1 is a diagram illustrating a configuration of an endoscope system 1 according to an embodiment.

The endoscope system 1 corresponds to a medical image processing system. The endoscope system 1 is a system used in the medical field to observe the inside of a living body. Then, as illustrated in FIG. 1, the endoscope system 1 includes an endoscope 2, a light source device 3, a display device 4, a control device 5, and a server 6.

The endoscope 2 corresponds to a medical observation device.

In the present embodiment, the endoscope 2 is a so-called flexible endoscope. The endoscope 2 is partially inserted into a living body, images the inside of the living body, and outputs an image signal obtained by imaging the inside of the living body. Then, as illustrated in FIG. 1, the endoscope 2 includes an insertion part 21, an operating unit 22, a universal cord 23, and a connector part 24.

The insertion part 21 is a portion at least partially flexible and inserted into a living body. As illustrated in FIG. 1, the insertion part 21 includes a distal end unit 211, a curved portion 212, and a flexible tube 213.

The distal end unit 211 is provided at a distal end of the insertion part 21. Although not specifically illustrated, a lighting optical system, an imaging optical system, and an imaging unit are provided in the distal end unit 211.

The lighting optical system faces one end of a light guide (not illustrated) routed inside the insertion part 21, and irradiates the inside of the living body with observation light transmitted by the light guide from the distal end of the insertion part 21.

The imaging optical system receives return light (subject image) of the observation light irradiated into the living body from the lighting optical system, and forms an image on an imaging surface of an imaging element constituting the imaging unit.

The imaging unit includes an imaging element such as a charge coupled device (CCD) or a complementary metal-oxide semiconductor (CMOS), captures a subject image formed by the imaging optical system, and outputs an image signal generated by capturing the subject image. Hereinafter, the image signal is referred to as imaging data.

The curved portion 212 is connected to a proximal end side (operating unit 22 side) of the distal end unit 211. Although not specifically illustrated, the curved portion 212 has a configuration in which a plurality of curved pieces are connected, allowing the curved portion 212 to be curved.

The flexible tube 213 is connected to a proximal end side (operating unit 22 side) of the curved portion 212, and is formed in an elongated shape with flexibility.

The operating unit 22 is connected to a proximal end portion of the insertion part 21. Then, the operating unit 22 receives various operations on the endoscope 2.

The universal cord 23 is a cord extending from the operating unit 22 in a direction different from the direction in which the insertion part 21 extends, in which the above-described light guide, a signal line for transmitting the above-described imaging data, and the like are arranged.

The connector part 24 is provided at an end portion of the universal cord 23, and is detachably connected to the light source device 3 and the control device 5.

In the present embodiment, the light source device 3 and the control device 5 are configured so that various endoscopes 2 of different release generations can be connected thereto. These various endoscopes 2 of different release generations are different in the type of imaging element (CCD or CMOS), the number of pixels of the imaging element, the sensitivity of the imaging element, the characteristics of the lighting optical system and the imaging optical system, etc.

The light source device 3 supplies observation light to the other end of the above-described light guide under the control of the control device 5. Then, the observation light passes through the above-described light guide and the above-described lighting optical system, and then is irradiated from the distal end of the insertion part 21 into a living body. Examples of the observation light include white light (normal light), which is wide band light, excitation light, which is narrow band light for exciting a fluorescent agent such as indocyanine green, and narrow band light used in narrow band imaging (NBI).

The display device 4 is a liquid crystal display (LCD), an organic electro luminescence (EL) display, or the like, and displays a display image (endoscopic image) corresponding to a video signal output from the control device 5 under the control of the control device 5.

The control device 5 corresponds to a medical image processing apparatus. The control device 5 includes a controller such as a central processing unit (CPU) or a micro processing unit (MPU), and controls operations of the endoscope 2 (the above-described imaging unit and the like) and the light source device 3 in an integrated manner. Note that the control device 5 is not limited to the CPU and the MPU, and may include an application specific integrated circuit (ASIC), a field-programmable gate array (FPGA), a graphics processing unit (GPU), and the like.

Note that a detailed configuration of the control device 5 will be described in the section “Configuration of Control Device” to be described later.

The server 6 communicates with the control device 5 via a network NE (FIG. 1). Note that the server 6 is realized by a single server in FIG. 1, but is not limited thereto, and may be realized by a cloud system in which a plurality of servers operate in cooperation.

Note that a detailed configuration of the server 6 will be described in the section “Configuration of Server” to be described later.

### Configuration of Control Device

Next, a configuration of the control device 5 will be described.

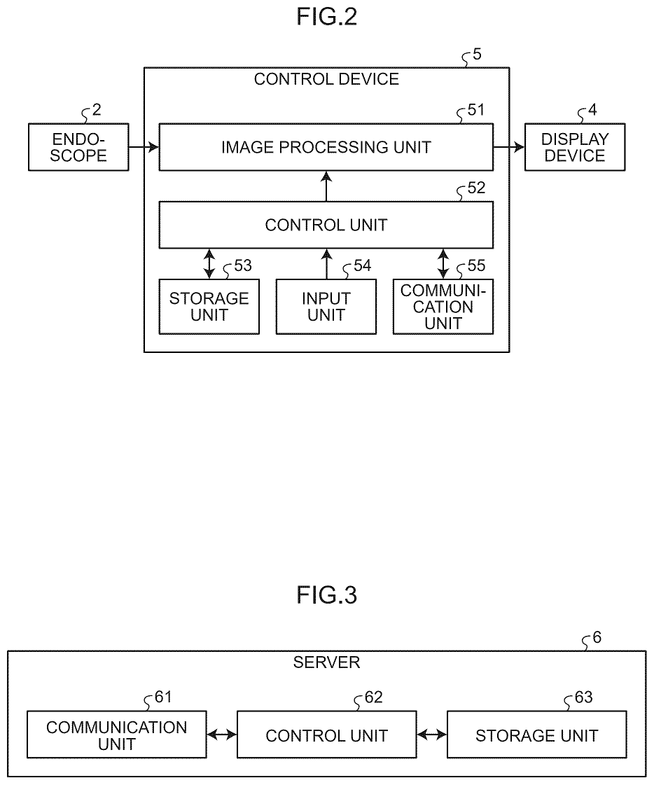

FIG. 2 is a block diagram illustrating a configuration of the control device 5.

As illustrated in FIG. 2, the control device 5 includes an image processing unit 51, a control unit 52, a storage unit 53, an input unit 54, and a communication unit 55.

The image processing unit 51 includes at least one processing module received via the communication unit 55. Then, the image processing unit 51 executes image processing on the imaging data (raw data) obtained by imaging using the endoscope 2 according to an image processing algorithm corresponding to the at least one processing module, and generates a video signal corresponding to a display image to be displayed on the display device 4.

Here, the processing module is a processing module serving as a minimum unit of each function constituting the image processing algorithm, which is also referred to as a microservice (hereinafter, referred to as MS). Note that the processing module according to the disclosure is not limited to the MS, and may be configured by combining a plurality of minimum units of functions constituting the image processing algorithm.

The image processing executed by the processing module includes noise removal, brightness correction (gain correction), and structure enhancement. Furthermore, the image processing executed by the processing module may include image processing according to observation light (white light, excitation light, or narrow band light) irradiated into the living body. Examples of the image processing according to observation light irradiated into the living body include optical black subtraction processing (clamping processing), white balance adjustment processing, demosaic processing, color correction matrix processing, gamma correction processing, and YC processing for converting an RGB signal into a luminance chrominance signal (Y, Cb/Cr signal).

The control unit 52 is realized by a controller such as a CPU and an MPU executing various programs stored in the storage unit 53, and controls the operations of the endoscope 2 (the above-described imaging unit and the like) and the light source device 3 and controls the overall operation of the control device 5. Note that the control unit 52 is not limited to the CPU and the MPU, and may include an ASIC, an FPGA, and the like. Then, the control unit 52 has functions as an information acquisition unit and a processing control unit according to the disclosure.

The functions of the control unit 52 as an information acquisition unit and a processing control unit will be described in the section “Operation of Endoscope System” to be described later.

The storage unit 53 stores programs to be executed by the control unit 52, information necessary for the processing of the control unit 52, and the like.

The input unit 54 is configured using operation devices such as a mouse, a keyboard, and a touch panel, and receives a user operation. Then, the input unit 54 outputs an operation signal corresponding to the user operation to the control unit 52.

The communication unit 55 communicates with the server 6 via the network NE under the control of the control unit 52.

### Configuration of Server

Next, a configuration of the server 6 will be described.

FIG. 3 is a block diagram illustrating a configuration of the server 6.

As illustrated in FIG. 3, the server 6 includes a communication unit 61, a control unit 62, and a storage unit 63.

The communication unit 61 communicates with the control device 5 (communication unit 55) via the network NE under the control of the control unit 62.

The control unit 62 is realized by a controller such as a CPU and an MPU executing various programs recorded in the storage unit 63, and controls the overall operation of the server 6. Note that the control unit 62 is not limited to the CPU and the MPU, and may include integrated circuits such as an ASIC and an FPGA.

Note that the functions of the control unit 62 will be described in the section “Operation of Endoscope System” to be described later.

The storage unit 63 stores various programs to be executed by the control unit 62, data required when the control unit 62 performs processing, and the like.

Note that the information stored in the storage unit 63 will be described in detail in the section “Operation of Endoscope System” to be described later.

### Operation of Endoscope System

Next, an operation of the endoscope system 1 will be described. The operation of the endoscope system 1 to be described below includes a medical image processing method according to the disclosure.

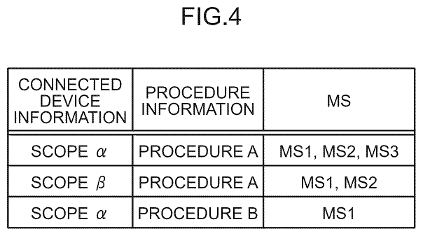

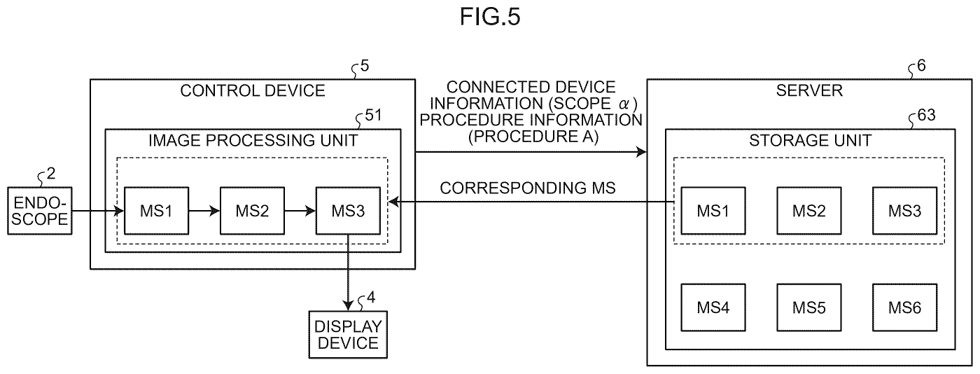

FIGS. 4 and 5 are diagrams illustrating an example of a medical image processing method. Specifically, FIG. 4 is a diagram illustrating information stored in the storage unit 63. FIG. 5 is a diagram corresponding to FIG. 4, and is a diagram illustrating information transmitted and received by communication between the control device 5 and the server 6. Note that, in FIG. 5, for convenience of explanation, only the image processing unit 51 is illustrated as the configuration of the control device 5, and only the storage unit 63 is illustrated as the configuration of the server 6.

Here, as illustrated in FIG. 4, the storage unit 63 stores connected device information specific to the endoscope 2 for identifying the endoscope 2, procedure information regarding procedure, and at least one MS in association with each other. In the example of FIG. 4, “scope α”, which is connected device information, “procedure A”, which is procedure information, and “MS1, MS2, and MS3”, which are three MSs, are stored in association with each other in the storage unit 63. The connected device information is a scope identifier (ID) of the endoscope 2, and is stored in a memory (not illustrated) provided in the endoscope 2.

For example, based on log information stored in a case where a specific MS was used for image processing when an operator or an inspector performed a specific procedure using a specific endoscope 2 in the past, connected device information specific to the endoscope 2, the procedure information regarding procedure, and the MS are stored in association with each other in the storage unit 63.

First, the control device 5 (control unit 52) reads a scope ID (connected device information) from the memory (not illustrated) of the endoscope 2 connected to the control device 5. In addition, the control unit 52 acquires procedure information regarding procedure input by a user operation on the input unit 54.

Next, the control unit 52 transmits, to the server 6 via the communication unit 55, the scope ID (connected device information) read from the memory (not illustrated) of the endoscope 2 and the procedure information input by the user operation on the input unit 54.

Next, the server 6 (the control unit 62) recognizes an MS associated with the scope ID (connected device information) and the procedure information received from the control device 5 with reference to the information stored in the storage unit 63. Then, the control unit 62 transmits the recognized MS to the control device 5 via the communication unit 61.

Next, the control device 5 (control unit 52 (information acquisition unit)) receives the MS from the server 6 (information acquisition step). Then, the control unit 52 (processing control unit) causes the image processing unit 51 to execute image processing on imaging data obtained by imaging using the endoscope 2 according to an image processing algorithm corresponding to the MS received from the server 6 (image processing step).

In the example of FIG. 5, “scope α” as connected device information and “procedure A” as procedure information are transmitted from the control device 5 to the server 6. As a result, the server 6 transmits “MS1, MS2, and MS3 (FIG. 4)” as MSs corresponding to the “scope α” and the “procedure A” to the control device 5. Then, in the control device 5, image processing is executed by “MS1”, “MS2”, and “MS3” in this order on the imaging data obtained by imaging using the endoscope 2, and a display image corresponding to the imaging data after the image processing is displayed on the display device 4.

According to the present embodiment described above, the following effects are obtained.

The control device 5 according to the present embodiment acquires at least one MS corresponding to connected device information and procedure information from the server 6, and executes image processing on imaging data obtained by imaging using the endoscope 2 according to an image processing algorithm corresponding to the at least one MS. Therefore, it is possible to execute appropriate image processing corresponding to the endoscope 2 connected to the control device 5 and the procedure on the imaging data obtained by imaging using the endoscope 2, and it is possible to display a display image suitable for observation on the display device 4. In addition, since the corresponding MS is acquired from the server 6 before performing the procedure, even if an abnormality occurs in communication between the control device 5 and the server 6 during performing the procedure, it is possible to continue to display the display image on the display device 4. As a result, the control device 5 according to the present embodiment is capable of suppressing image loss and improving convenience.

### OTHER EMBODIMENTS

Although the mode for carrying out the disclosure has been described so far, the disclosure should not be limited only by the above-described embodiment.

Configurations of modifications 1 to 6 to be described below may also be adopted.

### First Modification

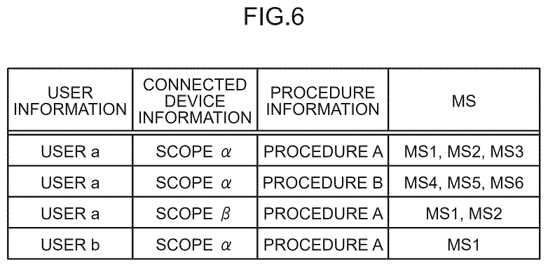

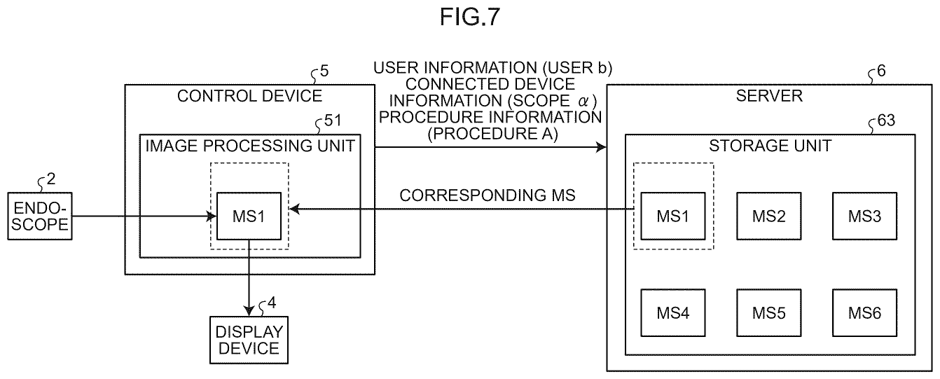

FIGS. 6 and 7 are diagrams illustrating a first modification of the embodiment. Specifically, FIG. 6 is a diagram illustrating information stored in the storage unit 63. FIG. 7 is a diagram corresponding to FIG. 6, and is a diagram illustrating information transmitted and received by communication between the control device 5 and the server 6. Note that, in FIG. 7, for convenience of explanation, only the image processing unit 51 is illustrated as the configuration of the control device 5, and only the storage unit 63 is illustrated as the configuration of the server 6.

In the above-described embodiment, the control device 5 (control unit 52 (information acquisition unit)) may acquire at least one MS corresponding to the connected device information, the procedure information, and the user information for identifying the user who uses the endoscope 2 from the server 6.

Here, as illustrated in FIG. 6, the storage unit 63 stores connected device information, procedure information, and user information in association with at least one MS. In the example of FIG. 6, “scope α”, which is connected device information, “procedure A”, which is procedure information, “user a”, which is user information, and “MS1, MS2, and MS3”, which are three MSs, are stored in association with each other in the storage unit 63.

For example, based on log information stored in a case where a specific MS was used for image processing when an operator or an inspector performed a specific procedure using a specific endoscope 2 in the past, the connected device information specific to the endoscope 2, the procedure information regarding procedure, and the user information for identifying the operator or the inspector are stored in association with the MS in the storage unit 63.

In the example of FIG. 7, “scope α” as connected device information, “procedure A” as procedure information, and “user a” as user information are transmitted from the control device 5 to the server 6. Here, the user information is information input by a user operation on the input unit 54. That is, the control device 5 acquires the user information input by the user operation on the input unit 54. As a result, the server 6 transmits “MS1 (FIG. 6)” as an MS corresponding to the “scope α”, the “procedure A”, and the “user a” to the control device 5. Then, in the control device 5, image processing is executed by “MS1” on the imaging data obtained by imaging using the endoscope 2, and a display image corresponding to the imaging data after the image processing is displayed on the display device 4.

According to the first modification described above, the following effects are obtained in addition to the effects that are the same as those of the embodiment described above.

The control device 5 according to the first modification acquires at least one MS corresponding to user information as well as connected device information and procedure information from the server 6, and executes image processing on imaging data obtained by imaging using the endoscope 2 according to an image processing algorithm corresponding to the at least one MS. Therefore, it is possible to execute image processing similar to the image processing used by the operator or the inspector in the past on the imaging data obtained by imaging using the endoscope 2, and it is possible to display a display image that looks the same as the display image observed in the past on the display device 4. As a result, convenience can be further improved.

### Second Modification

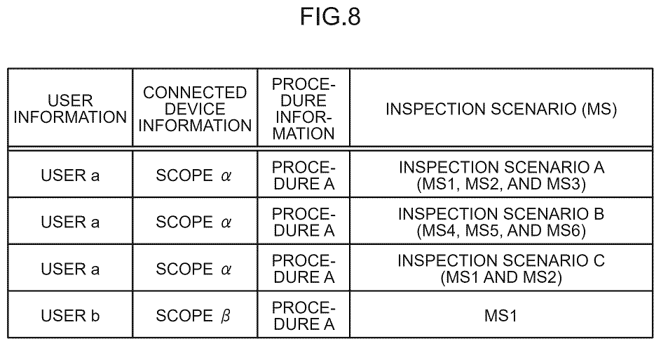

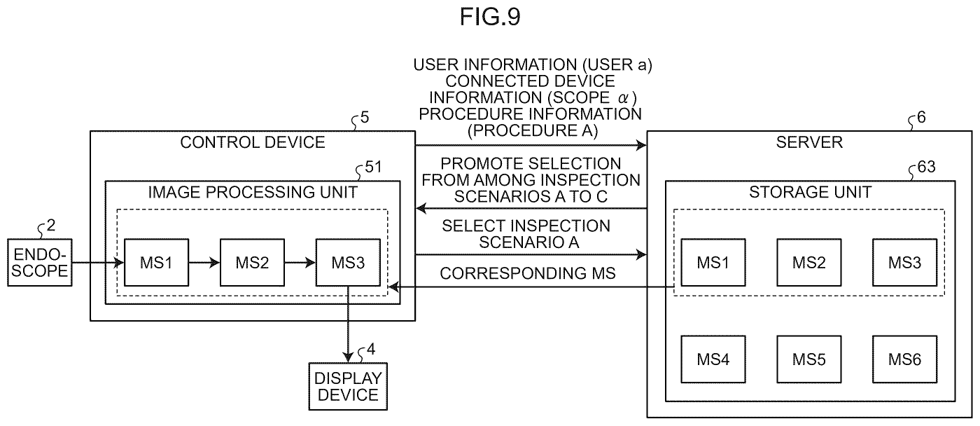

FIGS. 8 and 9 are diagrams illustrating a second modification of the embodiment. Specifically, FIG. 8 is a diagram illustrating information stored in the storage unit 63. FIG. 9 is a diagram corresponding to FIG. 8, and is a diagram illustrating information transmitted and received by communication between the control device 5 and the server 6. Note that, in FIG. 9, for convenience of explanation, only the image processing unit 51 is illustrated as the configuration of the control device 5, and only the storage unit 63 is illustrated as the configuration of the server 6.

In the first modification described above, a plurality of different MSs may be stored in the storage unit 63 in association with each set of connected device information, procedure information, and user information. In the example of FIG. 8, inspection scenarios A to C are stored in the storage unit 63 as MSs in association with a set of “scope α”, which is connected device information, “procedure A”, which is procedure information, and “user a”, which is user information.

The inspection scenario A is constituted by three MSs “MS1, MS2, and MS3”. The inspection scenario B is constituted by three MSs “MS4, MS5, and MS6”. The inspection scenario C is constituted by two MSs “MS1 and MS2”. In these inspection scenarios A to C, since the MSs are different, the way the display image appears after the image processing is performed by the MSs is different.

For example, based on log information stored in a case where a specific MS was used for image processing when an operator or an inspector performed a specific procedure using a specific endoscope 2 in the past, the connected device information specific to the endoscope 2, the procedure information regarding procedure, and the user information for identifying the operator or the inspector are stored in association with the MS in the storage unit 63. Furthermore, in a case where a set of connected device information, procedure information, and user information that is the same as the set stored in the storage unit 63 has already been stored in the storage unit 63, for example, MSs associated with the already stored set are stored in the storage unit 63 as inspection scenario A, and MSs associated with the set to be stored are stored in the storage unit 63 as inspection scenario B.

In the example of FIG. 9, “scope α” as connected device information, “procedure A” as procedure information, and “user a” as user information are transmitted from the control device 5 to the server 6. Here, as illustrated in FIG. 8, each of the inspection scenarios A to C is stored in the storage unit 63 in association with the set of “scope α”, “procedure A”, and “user a”. Therefore, the server 6 transmits, to the control device 5, information prompting selection from among the inspection scenarios A to C corresponding to the “scope α”, the “procedure A”, and the “user a”. In response thereto, when the inspection scenario A is selected by a user operation on the input unit 54, the control device 5 transmits information indicating that the inspection scenario A has been selected to the server 6. As a result, the server 6 transmits “MS1, MS2, and MS3 (FIG. 8)” to the control device 5 as MSs corresponding to the inspection scenario A. Then, in the control device 5, image processing is executed by “MS1”, “MS2”, and “MS3” in this order on the imaging data obtained by imaging using the endoscope 2, and a display image corresponding to the imaging data after the image processing is displayed on the display device 4.

According to the second modification described above, the following effects are obtained in addition to the effects that are the same as those of the embodiment and the first modification described above.

The control device 5 according to the second modification acquires, from the server 6, at least one MS corresponding to an inspection scenario selected by an operator or an inspector among a plurality of different inspection scenarios associated with a set of connected device information, procedure information, and user information. Then, the control device 5 executes image processing on imaging data obtained by imaging using the endoscope 2 according to an image processing algorithm corresponding to the at least one MS. Therefore, the operator or the inspector can select one of the plurality of inspection scenarios that are different in the way the display image appears, and observe the display image that appears in the preferred way on the display device 4. As a result, convenience can be further improved.

Note that, in the second modification, a plurality of different inspection scenarios are stored in the storage unit 63 in association with a set of connected device information, procedure information, and user information, but the disclosure is not limited thereto. The user information is not essential, and a plurality of different inspection scenarios may be stored in the storage unit 63 in association with a set of only connected device information and procedure information.

### Third Modification

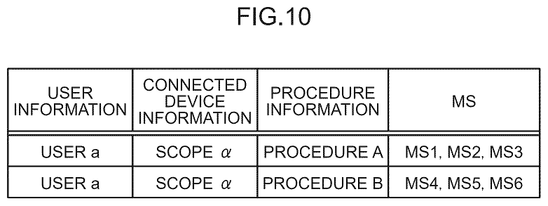

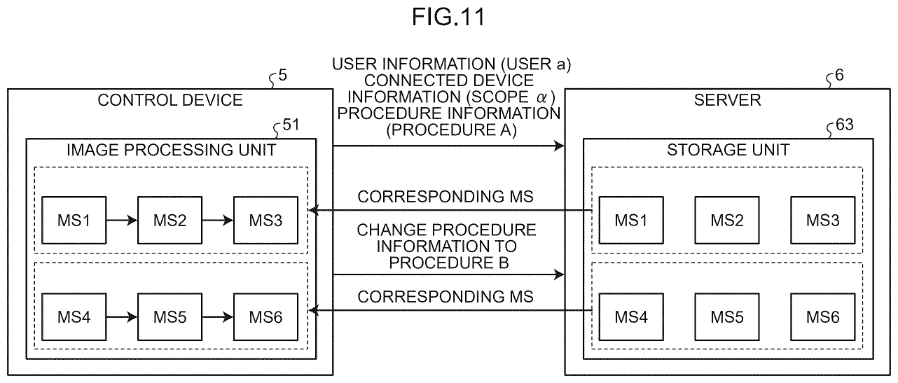

FIGS. 10 and 11 are diagrams illustrating a third modification of the embodiment. Specifically, FIG. 10 is a diagram illustrating information stored in the storage unit 63. FIG. 11 is a diagram corresponding to FIG. 10, and is a diagram illustrating information transmitted and received by communication between the control device 5 and the server 6. Note that, in FIG. 10, for convenience of explanation, only the image processing unit 51 is illustrated as the configuration of the control device 5, and only the storage unit 63 is illustrated as the configuration of the server 6.

In the above-described embodiment, for example, in a case where a specific procedure is planned to be performed before inspection, an MS corresponding to the specific procedure is acquired from the server 6. However, in a case where it is necessary to change the procedure information to a procedure different from the specific procedure during the inspection, an MS corresponding to the procedure after the change may be acquired from the server 6.

In the example of FIG. 11, first, “scope α” as connected device information, “procedure A” as procedure information, and “user a” as user information are transmitted from the control device 5 to the server 6. As a result, the server 6 transmits “MS1, MS2, and MS3 (FIG. 10)” as MSs corresponding to the “scope α”, the “procedure A”, and the “user a” to the control device 5. Then, in the control device 5, image processing is executed by “MS1”, “MS2”, and “MS3” in this order on the imaging data obtained by imaging using the endoscope 2, and a display image corresponding to the imaging data after the image processing is displayed on the display device 4. Here, in a case where the procedure information is changed from “procedure A” to “procedure B” during the inspection, the server 6 transmits “MS4, MS5, and MS6 (FIG. 10)” as MSs corresponding to the “scope α”, the “procedure B”, and the “user a”. As a result, the control unit 52 (information acquisition unit) acquires the “MS4, MS5, and MS6”. Then, the control unit 52 (processing control unit) causes image processing to be executed by “MS4”, “MS5”, and “MS6” in this order on imaging data obtained by imaging using the endoscope 2. As a result, a display image corresponding to the imaging data after the image processing is displayed on the display device 4.

According to the third modification described above, the following effects are obtained in addition to the effects that are the same as those of the embodiment described above.

When the procedure information is changed to a procedure different from the specific procedure during the inspection, the control device 5 according to the third modification acquires at least one MS corresponding to the procedure after the change from the server 6. Then, the control device 5 executes image processing on imaging data obtained by imaging using the endoscope 2 according to an image processing algorithm corresponding to the at least one MS. That is, since the image processing corresponding to the procedure after the change is executed on the imaging data obtained by the imaging using the endoscope 2, it is possible to display a display image suitable for observation on the display device 4. As a result, convenience can be further improved.

### Fourth Modification

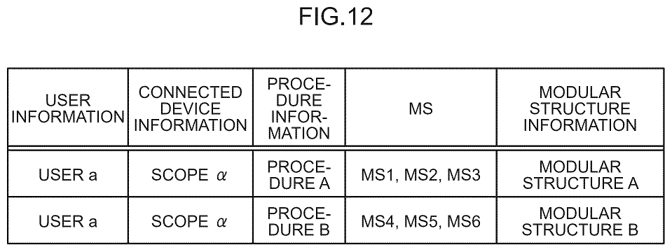

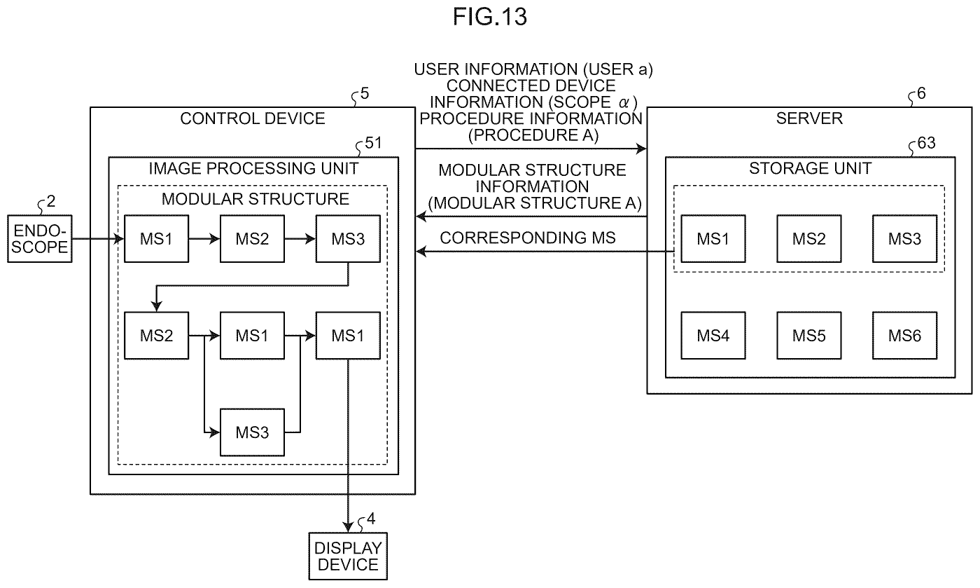

FIGS. 12 and 13 are diagrams illustrating a fourth modification of the embodiment. Specifically, FIG. 12 is a diagram illustrating information stored in the storage unit 63. FIG. 13 is a diagram corresponding to FIG. 12, and is a diagram illustrating information transmitted and received by communication between the control device 5 and the server 6. Note that, in FIG. 13, for convenience of explanation, only the image processing unit 51 is illustrated as the configuration of the control device 5, and only the storage unit 63 is illustrated as the configuration of the server 6.

In the above-described embodiment, a modular structure may be configured by combining a plurality of MSs, and image processing may be executed on imaging data according to an image processing algorithm corresponding to the modular structure.

Here, as illustrated in FIG. 12, the storage unit 63 stores connected device information, procedure information, and user information in association with a plurality of MSs and modular structure information. The modular structure information is information for combining the plurality of MSs to configure a modular structure. In the example of FIG. 12, “scope α”, which is connected device information, “procedure A”, which is procedure information, “user a”, which is user information, are stored in the storage unit 63 in association with “MS1, MS2, and MS3”, which are three MSs, and “modular structure A”, which is modular structure information.

For example, based on log information stored in a case where a specific modular structure was used for image processing when an operator or an inspector performed a specific procedure using a specific endoscope 2 in the past, the connected device information specific to the endoscope 2, the procedure information regarding procedure, and the user information for identifying the operator or the inspector are stored in the storage unit 63 in association with a plurality of MSs constituting the modular structure, and modular structure information for combining the plurality of MSs to configure the modular structure.

In the example of FIG. 13, “scope α” as connected device information, “procedure A” as procedure information, and “user a” as user information are transmitted from the control device 5 to the server 6. As a result, the server 6 transmits “MS1, MS2, and MS3 (FIG. 12)” and “modular structure A (FIG. 12)” as MSs and modular structure information corresponding to the “scope α”, the “procedure A”, and the “user a” to the control device 5. As a result, the control unit 52 (information acquisition unit) acquires the “MS1, MS2, and MS3” and the “modular structure A”. Furthermore, the control unit 52 (processing control unit) configures a modular structure (FIG. 13) by combining the “MS1, MS2, and MS3” based on the “modular structure A”. Then, the control unit 52 (processing control unit) causes image processing to be executed on imaging data obtained by imaging using the endoscope 2 according to an image processing algorithm corresponding to the modular structure. As a result, a display image corresponding to the imaging data after the image processing is displayed on the display device 4.

According to the fourth modification described above, the following effects are obtained in addition to the effects that are the same as those of the embodiment described above.

The control device 5 according to the fourth modification acquires, from the server 6, a plurality of MSs and modular structure information for combining the plurality of MSs to configure a modular structure. Then, the control device 5 configures a modular structure based on the plurality of MSs and the modular structure information, and executes image processing on imaging data obtained by imaging using the endoscope 2 according to an image processing algorithm corresponding to the modular structure. Therefore, modular structures of various patterns can be configured. In addition, the amount of data transmitted from the server 6 to the control device 5 can be reduced.

In the fourth modification, the modular structure is configured on the control device 5 side, but the disclosure is not limited thereto, and the modular structure may be configured on the server 6 side.

### Fifth Modification

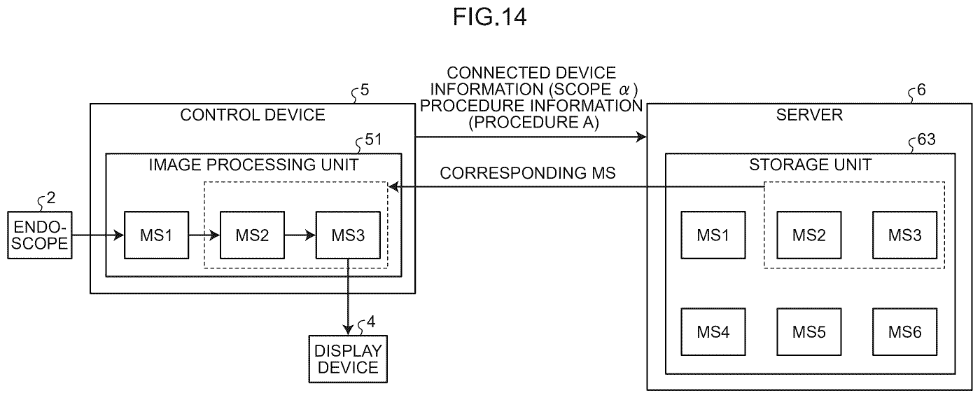

FIG. 14 is a diagram illustrating a fifth modification of the embodiment. Specifically, FIG. 14 is a diagram corresponding to FIG. 4, and is a diagram illustrating information transmitted and received by communication between the control device 5 and the server 6. Note that, in FIG. 14, for convenience of explanation, only the image processing unit 51 is illustrated as the configuration of the control device 5, and only the storage unit 63 is illustrated as the configuration of the server 6.

In the above-described embodiment, the image processing unit 51 may always include a minimum required MS. As the minimum required MS, image processing corresponding to white light irradiated into the living body can be exemplified.

In the example of FIG. 14, the image processing unit 51 always includes “MS1” as the minimum required MS. Then, in the example of FIG. 14, “scope α” as connected device information and “procedure A” as procedure information are transmitted from the control device 5 to the server 6. As a result, the server 6 transmits “MS2 and MS3” other than the “MS1” included in the image processing unit 51 among “MS1, MS2, and MS3 (FIG. 4)”, which are MSs corresponding to the “scope α” and the “procedure A”, to the control device 5. Then, in the control device 5, image processing is executed by “MS1”, “MS2”, and “MS3” in this order on the imaging data obtained by imaging using the endoscope 2, and a display image corresponding to the imaging data after the image processing is displayed on the display device 4.

According to the fifth modification described above, the following effects are obtained in addition to the effects that are the same as those of the embodiment described above.

In the fifth modification, the image processing unit 51 always includes a minimum required MS. Therefore, for example, in a case where image processing corresponding to white light irradiated into the living body is executed, the control device 5 does not need to acquire a minimum required MS from the server 6 through communication. In addition, since the control device 5 always includes a minimum required MS, in a case where image processing including the minimum required MS and another MS is executed, the amount of data transmitted from the server 6 to the control device 5 can also be reduced.

### Sixth Modification

In the embodiment and the first to fifth modifications described above, the medical image processing apparatus according to the disclosure is mounted on the endoscope system 1 in which the endoscope 2 is a flexible endoscope, but the disclosure is not limited thereto. For example, the medical image processing apparatus according to the disclosure may be mounted on an endoscope system in which the endoscope 2 is a rigid endoscope. In addition, the medical image processing apparatus according to the disclosure may be mounted on a medical observation system including a surgical microscope (see, for example, JP 2016-42981 A) that enlarges and observes a predetermined field of view inside a subject (inside a living body) or on a surface of a subject (on a surface of a living body). In this case, the surgical microscope corresponds to the medical observation device.

The medical image processing apparatus, the medical image processing system, the server, and the medical image processing method according to the disclosure are capable of improving convenience.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical image processing apparatus comprising:
a processor comprising hardware, the processor being configured to:
acquire, from an external server, at least one processing module corresponding to connected device information specific to a connected medical observation device for identifying the medical observation device and procedure information regarding procedure; and
execute image processing on imaging data obtained by imaging using the medical observation device according to an image processing algorithm corresponding to the at least one processing module.

2. The medical image processing apparatus according to claim 1, wherein
the processor is configured to
acquire, from the external server, the at least one processing module corresponding to the connected device information, the procedure information, and user information for identifying a user who uses the medical observation device.

3. The medical image processing apparatus according to claim 1, wherein
the processor is configured to:
additionally acquire, from the server, at least one processing module corresponding to the connected device information and changed procedure information; and
execute the image processing on the imaging data according to an image processing algorithm corresponding to the additionally acquired at least one processing module.

4. The medical image processing apparatus according to claim 1, wherein
the processor is configured to:
acquire, from the server, a plurality of processing modules and modular structure information for combining the plurality of processing modules to configure a modular structure; and
configure a modular structure by combining the plurality of processing modules based on the plurality of processing modules and the modular structure information, and execute the image processing on the imaging data according to an image processing algorithm corresponding to the modular structure.

5. The medical image processing apparatus according to claim 1, wherein
the processor includes a minimum necessary processing module.

6. The medical image processing apparatus according to claim 1, wherein
the processing module is a processing module that is a minimum unit of each function constituting the image processing algorithm.

7. A medical image processing system comprising:
a server; and
the medical image processing apparatus according to claim 1, the medical image processing apparatus being communicably connected to the server.

8. A server comprising:
a communicator communicably connected to an external medical image processing apparatus;
a controller configured to control an operation of the communicator; and
a storage configured to store connected device information specific to a medical observation device for identifying the medical observation device and procedure information regarding procedure in association with at least one processing module,
wherein the controller is configured to
read, from the storage, the at least one processing module associated with the connected device information and the procedure information acquired from the medical image processing apparatus via the communicator, and transmit the at least one processing module to the medical image processing apparatus via the communicator.

9. A medical image processing method executed by a medical image processing apparatus, the medical image processing method comprising:
acquiring, from an external server, at least one processing module corresponding to connected device information specific to a medical observation device for identifying the medical observation device connected to the medical image processing apparatus and procedure information regarding procedure; and
executing image processing on imaging data obtained by imaging using the medical observation device according to an image processing algorithm corresponding to the at least one processing module.

10. A non-transitory computer-readable recording medium with an executable program stored thereon, the program causing a processor of a medical image processing apparatus to execute:
acquiring, from an external server, at least one processing module corresponding to connected device information specific to a medical observation device for identifying the medical observation device connected to the medical image processing apparatus and procedure information regarding procedure; and
executing image processing on imaging data obtained by imaging using the medical observation device according to an image processing algorithm corresponding to the at least one processing module.
